(19) United States
(12) Patent Application Publication
(10) Pub. No.: US 2010/0249652 A1
(43) Pub. Date: Sep. 30, 2010

(54) Integrated Analyte Devices and Processes

(75) Inventors: Benjamin Rush, Oakland, CA (US); Kenneth J. Doniger, Menlo Park, CA (US)

Correspondence Address: JACKSON & CO., LLP, 6114 LA SALLE AVENUE, #507, OAKLAND, CA, 94611-2802, US

(73) Assignee: Abbott Diabetes Care Inc., Alameda, CA (US)

(21) Appl. No.: 12/752,097

(22) Filed: Mar. 31, 2010

Related U.S. Application Data
(60) Provisional application No. 61/165,494, filed on Mar. 31, 2009.
(60) Provisional application No. 61/165,503, filed on Mar. 31, 2009.

(51) Int. Cl. A61B 5/151

(52) U.S. Cl. 600/583

(57) ABSTRACT

Devices and processes for the analysis of a body fluid are provided. Embodiments include integrated devices that include two or more analyte testing components. Also provided are processes of analyte testing devices.

### RELATED APPLICATIONS

The present application claims priority under 35 U.S.C. §119(e) to U.S. provisional application No. 61/165,494 filed Mar. 31, 2009 entitled “Integrated Analyte Devices and Processes”, and U.S. provisional application No. 61/165,503 filed Mar. 31, 2009 entitled “Skin Piercing Devices”, the disclosures of which each of which are incorporated herein by reference in its entirety for all purposes.

### BACKGROUND

Analytical testing devices, e.g., test strips, for testing outside a clinical laboratory setting such as for home use are commonly used to determine an analyte level in biological fluid (the presence and/or concentration). Such test strips may be used, for example, to monitor blood glucose levels of diabetic patients.

In using analyte test strips, an opening is created in the skin (e.g., by puncturing the skin using a lancing device, for example) to cause a flow of biological fluid from the region. At least a portion of this biological fluid is contacted to the test strip where a feature of analyte in the fluid is then determined by reading the test strip with a test strip reader (also referred to as a meter) such as the presence and/or concentration of the analyte.

Typically, a number of different devices are used to perform an analyte test and include a skin piercing member or a lancing device, for example, a test strip, and a meter to read the test strip and provide results of the analyte test. These various components can be cumbersome to carry and use, and do not allow discrete testing that is often desired by many who test for analytes such as glucose. Furthermore, many people performing such analyte testing may have one or more physical impairments such as dexterity and/or vision problems that may make using multiple devices difficult, which may result in decreased testing compliance. Non compliance may also be attributed to simply failing to test at regular or prescribed times due to forgetfulness, e.g., in children or elderly, etc., or intentionally.

Lancing devices used to puncture the skin are usually configured to only include one lancet at a time so that a user must “load” a lancet into the lancing device each time the lancing device is used. Once used, a user must then either discard the lancing device with used lancet, or remove the used lancet from the lancing device which may have safety issues for the user or others who may come into contact with the used lancet. While some devices have been developed to include a plurality of lancets, these devices are complex, and oftentimes expensive to manufacture, and may still require a user to remove and discard used lancets. For devices that only include one lancet at a time, a user must carry un-used lancets for testing. Further, loading and removing a lancet for each test may be difficult, which may result in decreased testing compliance. Non compliance may also be attributed to simply failing to test at regular or prescribed times due to forgetfulness, e.g., in children or elderly, etc., or intentionally, or due to not having another fresh lancet or test strips on hand.

In instances that require analyte testing after skin has been lanced and biological fluid present at the lanced area, additional analyte testing devices are necessary, e.g., test strip and analyte meter to determine one or more analyte parameters from a sample of the biological fluid applied to a test strip. Carrying multiple components for analyte testing at all times is not desirable.

Accordingly, as analyte testing devices continue to be of importance in health management, there continues to be an interest in devices and methods that make testing easier, more discrete, and facilitates testing compliance. Of particular interest are analyte testing devices and methods that are easy and cost effective to manufacture, easy to use, and that may contain two or more analyte testing components as a single unit, e.g., in a single housing.

### SUMMARY

The subject disclosure provides devices and methods for analyte testing such as analyte concentration determination in a biological fluid sample. Embodiments of the subject disclosure include glucose testing devices for use outside of a clinical laboratory environment—for everyday or home use—that combine two or more glucose monitoring components together as an integrated unit, e.g., in a single, compact, hand-holdable, housing. Also provided are processes of analyte testing.

Embodiments of the subject devices include a housing having an interior space configured to house two or more analyte testing components. The housing may be adapted to be placed on a skin surface (e.g., discretely positioned on the body of a user such as easily hidden beneath clothing) and held in place for a period of time, e.g., so that multiple tests may be performed over a period of time. In certain embodiments an interior of a housing may include at least one analyte test strip and at least one skin piercing member, where embodiments include a plurality of test strips and/or one or more skin piercing members. A housing may include an axis such as a central axis, or the like, and positioned about the axis and/or which may be rotatable about the axis, may be a plurality of analyte test strips, e.g., radially positioned. One or more skin piercing members may be positioned about the axis and/or may be rotatable about the axis. In some embodiments, the one or more test strips are positioned in fixed relation, i.e., stationary, with respect to the axis, and the one or more skin piercing members may be moveable, e.g., rotatable, about the axis and moveable with respect to the one or more stationary test strips.

Some or all of the methods described herein may be computer implemented methods. For example, computer implemented methods may execute an action with or without user intervention, including user initiation. In some embodiments, a user need only confirm an action or recommendation of the computer, before or after execution thereof.

Devices may include hardware and/or software, e.g., one or more processors, to perform, or at least notify a user to perform, one or more analyte tests according to a predetermined schedule, e.g., a daily or weekly schedule. For example, a timing system may be coupled to one or more components of the device that may be pre-programmed or may be programmable (by a user or user's healthcare provider), to carry out such tasks, e.g., automatically so that one or more tests is executed without a user having to remember to execute the test. Accordingly, embodiments of the subject devices may be adapted to test a user's analyte level without any (or with little—for example a user may be required to confirm the execution of a test prior to its execution) user intervention, i.e., it may be automated, according to a testing schedule.

Embodiments of the subject disclosure include lancing devices that contain more than one lancet in an interior space of the lancing device. Also provided are methods of piercing skin to obtain a biological fluid sample expressed from pierced skin, and devices and methods of analyte testing.

Embodiments of the subject devices include a lancing device housing having an interior space. Within the interior space are at least two lancets and an advancement mechanism coupled to the two or more lancets to advance the lancets within the interior space, including advancing (and retracting in certain embodiments) a lancet into position to lancet skin through an opening in the housing. In certain embodiments, the housing also contains (or is coupleable to) analyte testing components such as an analyte test device (e.g., test strip) and/or electronics to perform an analyte test and read the results of the test, etc. Accordingly, an analyte tester and meter may also be integrated into the housing of a lancing device, or otherwise coupled thereto. In this manner, embodiments may include all of the components necessary to perform an analyte test, or multiple analyte tests, in a single containment unit.

In certain embodiments, the one more skin piercing members may include a cap that may be manually or automatically capped and/or re-capped from/to the skin piercing member.

An analyte meter may also be integrated in the interior of the housing, or otherwise coupled thereto. In this manner, embodiments may include all of the components necessary to perform an analyte test, or multiple analyte tests, in a single containment unit.

Many embodiments are calibration-free. In other words, no calibration action may be required by the user of the device.

The housing may be coupled to, e.g., include a reporting module such as a display or the like to report analyte results to a user. However, the housing may be devoid of a reporting module. In such embodiments (or in embodiments that include a reporting module), the housing may include a transmitter to transmit analyte test results to a remote receiving device which may include a reporting module such as a display to report analyte results to a user. In this way, discrete testing may be even more fully realized. In some embodiments, the housing may be substantially free—including completely free—of user interface components such as displays, buttons, etc., so that the sizing of the housing may be minimal and low profile.

Aspects of the tests strips of the subject disclosure include optical and electrochemical sensors. In many embodiments, the test strips are small-volume sensors. Small-volume sensors include sensors adapted to determine analyte concentration in a sample having a volume of about 1 microliter or less.

Also provided are methods of analyte testing, including computer-implemented methods of analyte testing.

### INCORPORATION BY REFERENCE

The following patents, applications and/or publications are incorporated herein by reference for all purposes: U.S. Pat. Nos. 5,264,104; 5,356,786; 5,262,035; 5,320,725; 6,990,366; 7,381,184; 7,299,082; 7,167,818; 7,041,468; 6,942,518; 6,893,545; 6,881,551; 6,773,671; 6,764,581; 6,749,740; 6,746,582; 6,736,957; 6,730,200; 6,676,816; 6,618,934; 6,616,819; 6,600,997; 6,592,745; 6,591,125; 6,560,471; 6,540,891; 6,514,718; 6,514,460; 6,503,381; 6,461,496; 6,377,894; 6,338,790; 6,299,757; 6,284,478; 6,270,455; 6,175,752; 6,161,095; 6,144,837; 6,143,164; 6,134,461; 6,121,009; 6,120,676; 6,071,391; 5,918,603; 5,899,855; 5,822,715; 5,820,551; 5,628,890; 5,601,435; 5,593,852; 5,509,410; 5,320,715; 5,264,014; 5,262,305; 5,262,035; 4,711,245; and 4,545,382; and U.S. Publication Nos. 2009/0018425; 2009/0054749; 2009/0257911 A1; 2009/0281406; 2009/0294277; 2008/0058625; 2008/0064937 A1; 2008/0071157; 2008/0071158; 2008/0179187; 2008/0319295; 2008/0319296; 2007/0149873; 2007/0149875; 2009/0321277; 2010/0030052; and 2004/0186365; and U.S. patent application Ser. Nos. 12/211,014 filed Sep. 15, 2008; 12/242,780 filed Sep. 30, 2008; 12/393,921 filed Feb. 27, 2009; 12/495,709 filed Jun. 30, 2009; 12/495,712 filed Jun. 30, 2009; 12/495,730 filed Jun. 30, 2009; 12/544,061 filed Aug. 19, 2009; Ser. No. 12/625,185 filed Nov. 24, 2009; 12/625,208 filed Nov. 24, 2009; Ser. No. 12/625,524 filed Nov. 24, 2009; Ser. No. 12/625,525 filed Nov. 24, 2009; Ser. No. 12/625,528 filed Nov. 24, 2009; Ser. No. 12/624,767 filed Nov. 24, 2009; Ser. No. 12/628,177 filed Nov. 30, 2009; Ser. No. 12/628,198 filed Nov. 30, 2009; Ser. No. 12/628,201 filed Nov. 30, 2009; Ser. No. 12/628,203 filed Nov. 30, 2009; Ser. No. 12/628,210 filed Nov. 30, 2009; Ser. No. 12/698,129 filed Feb. 1, 2010; Ser. No. 12/698,124 filed Feb. 1, 2010; Ser. No. 12/699,653 filed Feb. 3, 2010; Ser. No. 12/699,844 filed Feb. 3, 2010; and Ser. No. 12/714,439 filed Feb. 26, 2010; and U.S. Provisional Patent Application No. 61/238,646 filed Aug. 31, 2009.

### BRIEF DESCRIPTION OF THE DRAWINGS

FIG. 1 is a view of an embodiment of an analyte test device according to the subject disclosure;

FIGS. 2A and 2B show embodiments of devices, and methods of sterilization, of skin piercing members according to the subject disclosure;

FIGS. 3A and 3B show an exemplary embodiment of a lancing device according to the subject disclosure, wherein FIG. 3A shows a front view and FIG. 3B shows a cut-away view; and

FIG. 4 shows a detailed view of the lancet roller chain assembly of FIGS. 3A and 3B.

To facilitate understanding, identical reference numerals have been used, where practical, to designate the same elements which are common to different figures. The figures shown herein are not necessarily drawn to scale, with some components and features being exaggerated for clarity.

When two or more items (for example, elements or processes) are referenced by an alternative “or”, this indicates that either could be present separately or any combination of them could be present together except where the presence of one necessarily excludes the other or others.

Any recited method can be carried out in the order of events recited or in any other order which is logically possible. Reference to a singular item, includes the possibility that there are plural of the same item present.

### DETAILED DESCRIPTION

Before the present disclosure is described, it is to be understood that this disclosure is not limited to particular embodiments described, as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges is also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described. All publications mentioned herein are incorporated herein by reference to disclose and describe the methods and/or materials in connection with which the publications are cited.

It must be noted that as used herein and in the appended claims, the singular forms “a”, “an”, and “the” include plural referents unless the context clearly dictates otherwise.

The publications discussed herein are provided solely for their disclosure prior to the filing date of the present application. Nothing herein is to be construed as an admission that the present disclosure is not entitled to antedate such publication by virtue of prior disclosure. Further, the dates of publication provided may be different from the actual publication dates which may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure.

As summarized above, embodiments of the present disclosure include analyte (e.g., glucose) testing devices that have at least one skin piercing member and at least one test strip that are integrated in the interior space of a housing. The devices may be programmed or may be programmable to be fully or semi-automated devices to obtain a biological sample from a user at predetermined times, e.g., equal or unequal time intervals, without any action on the part of the user, and to test the obtained sample for analyte. Accordingly, embodiments include devices that include programming (or that may be programmable) to obtain glucose time profiles of a user, without the user having to actively or consciously initiate each test, using the subject devices. The time profiles of the data obtained from one or more tests may include less data points than achievable with continuous monitoring systems, but which may be less complicated and require less user training than certain continuous monitoring systems. One or more microprocessors may include programming to determine time profiles may be determined dynamically, e.g., accordingly to prior history of a user, or the time profiles may be static.

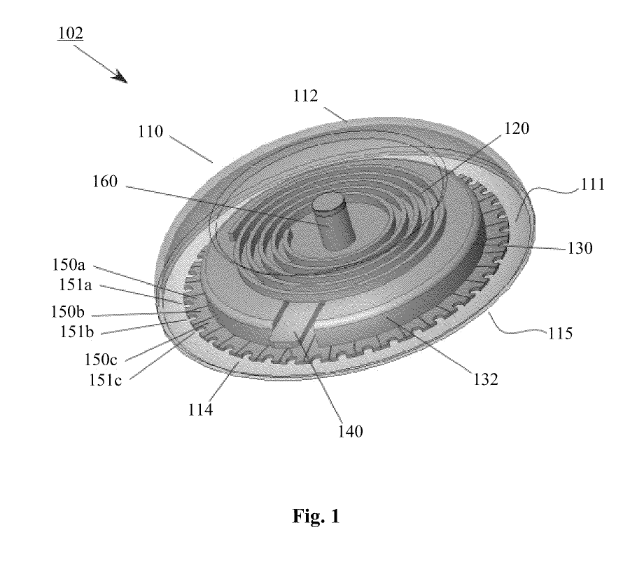

FIG. 1 shows one embodiment of analyte testing device 102 that includes a housing 110 that defines interior space 111. Housing 110 may include one or more parts, e.g., two or more parts, herein shown as including cover 112 and base 114 that is in the form of a stationary disk. Cover 112 of housing 110 is shown in FIG. 1 as a dome shape and encompasses test strip disk 114 having one or more test strips 150a, 150b, 150c . . . , which test strips radiate from a hub 160. It is to be understood that the dome-like shape is merely exemplary and any suitable housing shape may be employed. For example, housing 110 may be configured to be a substantially flat disk, e.g., similar to a hockey puck shape. In other embodiments, housing 110 may be rectangularly-shaped which may make the device smaller and more discrete in certain instances. A rectangularly-shaped housing may be desirable to house a fewer number of test strips than the dome shape.

One or more of the test strips of the housing may be located about the perimeter of thin disk 114. Disk 114 is fixed to hub 160 and may be stationary relative to the hub, which may be a central hub (i.e., may be substantially positioned about a central axis of the device). In certain embodiments, disk 114 may be rotatable about hub 160. Testing sites are defined by opening(s) (not shown) through disk 114 where biological fluid is collected-each opening corresponding to, and aligned with, a respective test strip. In some embodiments, portions of the device may be re-usable and portions may be one time use, e.g., discardable or consumable. For example, the one or more test strips (e.g., used or un-useable), and/or in certain instance the disk, and/or the one or more skin piercing members, may be removable from the housing and discarded. The device may be adapted so that replacement components may be easily added, e.g., after manufacture such as by a user or healthcare provider. In this manner, cost of the device is minimized as more complex and/or expensive portions (e.g., electronics, etc.,) may be reused, while other less expensive portions may be replaceable, thereby amortizing the cost of such a device over a longer period than would be if the entire device had to be discarded after exhausting a given supply of consumable items, such as test strips, of the device.

Each test strip 150a, 150b, 150c includes sample application area 151a, 151b, 151c adapted to receive an expressed sample of biological fluid into a sample chamber of the test strip for testing for analyte. Each of the sample application areas herein shown as 151a, 151b, 151c . . . with respect to tests strips 150a, 150b, and 150c, is also aligned or alignable with an opening of the disk 114. The sample application area of a strip may be positioned at the front edge of a strip as shown in FIG. 1, i.e., the test strips may be front perimeter fill strips, or may be positioned elsewhere, e.g., may be side perimeter fill, may be a corner fill test strip, or may be located at a site that is not a perimeter edge or corner of a strip, e.g., may be a top fill test strip, where “top” is used in a relative sense. The sample application area of a test strip may be generally positioned at an approximate proximal end of a strip and an approximate distal end may be generally configured to connect to, and provide electrical communication with, electronics, i.e., a meter, for reading the strip. Test strips that that may be filled with sample from more than one side of the strip and/or which may detect sample filling regardless of from which side the sample chamber is filled may be used and include strips having overhangs (e.g., cantilever test strips), as well as partial fill test strips, and test strips that include fill assist tabs/protrusions, all of which are particularly well suited for the analyte test devices described herein, such as those described in e.g., U.S. patent application Ser. Nos. 11/225,659; 11/237,447; and 11/277,931, the disclosures of which are herein incorporated by reference. Various other features and properties of test strips that may be employed in the subject devices are described below.

Device 102 may be stationarily attached to the body of the user, i.e., attached to a user in a substantially fixed position, such as attachable to a skin surface so that the bottom surface of the disk is positioned against a skin surface of a user. A device may be adapted to be fixed in position on the body of a user for a period of time, e.g., at least about 8 hours, e.g., at least about 24 hours, e.g. one day or more, e.g., one week or more, e.g., one month or more. Such an on-body device may be affixed to the skin in any suitable manner. For example, it may be fastened in place by an adhesive, it may be held in place by a strap, e.g., an arm band, wrist band, leg band, abdomen band, etc., or it could be configured to give the user the option to use any one or combination of ways to hold the device in place. For example, the device may include both an adhesive backing and a detachable strap such as an arm band. FIG. 1 shows adhesive surface 115 for attaching to a user. The adhesive may be applied to the surface of disk 114, or may be part of a mounting unit (not shown) that is configured to receive device 102. Suitable mounting units are described, e.g., in U.S. Pat. No. 6,175,752, the disclosure of which is herein incorporated by reference, and elsewhere. Embodiments of the disclosure are particularly suited for attachment to an alternative testing site of a user (a body site other than a finger tip, e.g., a forearm, leg, abdomen, etc.), description of which can be found in, among others, U.S. Pat. No. 7,299,082 and U.S. patent application Ser. No. 12/257,381, the disclosures of each of which are incorporated in their entirety by reference for all purposes. Accordingly, embodiments that include more than one test strip are configured to perform multiple analyte tests without displacement of the housing of the device from a given location on a body of a user, where such multiple tests may be executed automatically. For example, embodiments include microprocessor-executable modes such that some or all steps of analyte testing are carried out by suitable hardware and/or software without any, or with little, action by a user of the device to perform one or more analyte tests, including respective skin piercing.

Also inside interior space 111 is skin piercing member holder 130 to hold at least one skin piercing member 140 that is aligned or is alignable with an opening of the disk to pierce through the opening of the disk to the skin of a user. In many embodiments, holder 130 is rotationally attached to hub 160, although in certain embodiments it could be non-rotatable, e.g., stationary, with respect to hub 160. It may be desirable not to test multiple times at the exact same location. Accordingly, a skin piercing member may be configured to move, e.g., rotate, between test sites (between test strips of the device), where in many embodiments the axis of rotation is hub 160. For example, if holder 130 includes fewer skin piercing members than the number of test strips of disk 114, holder 130 may be rotatable about hub 160 to advance a skin piercing member to a certain test strip to conduct a test. If the same or greater number of skin piercing members than test strips is included, then holder 130 may not be rotatable. In the embodiments in which holder 130 is configured to rotate about an axis of rotation defined by the device, holder 130 may be rotatable about fixed hub 160 in discrete steps. For example, holder 130 may rotate and advance one step for each test, and each test may correspond to the distance between adjacent test sites, i.e., adjacent test strips. Any suitable skin piercing member may be used, where cantilever skin piercing members are particularly advantageous given the angle of the device with respect to a given test site.

In certain embodiments, the skin piercing member(s) is re-sterilizable, and in many embodiments re-sterilizable and self-sterilizable. In this manner, fewer number of skin piercers than test strips may be included, and a given skin piercer may be used for more than one analyte test while mitigating the risk of infection. For example, a single skin piercing member may be sterilized multiple times (re-sterilized) and used with multiple test strips of the device for multiple tests. In certain embodiments, a single skin piercer may be configured to advance (e.g., automatically under the control of a timing mechanism) to a plurality of different test strips and may be used with each test strip to obtain sample for a test, where the number of test strips that may be provided may be sufficient for one or more tests for multiple days or weeks. In certain embodiments, the number of test strips may range from about 1 to about 100 or more, and the number of skin piercers may range from about 1 to about 100 or more, e.g., the number of test strips may range from about 1 to about 100 or more, and the number of skin piercers may be 1. In some embodiments, one or more skin piercers may include covers at least over the sharp portion of the skin piercing member. Covers may be automatically moved or removed by the device or may be pierceable by the skin piercing member so that removal is not necessary. In certain embodiments, covers may include a cover removing and/or replacing mechanism contained within the interior space of the housing that maintains a removed cover within the housing. For example, a cover removing and/or replacing mechanism may remove a cover from a skin piercer and maintain it within the housing, the skin piercer may be used to pierce, and the cover may be replaced on the used skin piercer. In other embodiments, a cover may be removed from a skin piercer and ejected from the device. Covers for skin piercing members that may be employed in certain embodiments of the subject disclosure include, but are not limited to, those described in U.S. patent application Ser. No. 11/396,355, the disclosure of which is herein incorporated by reference.

In certain embodiments in which the skin piercing member(s) 140 is sterilizable from within the housing, e.g., re-sterilizable, the sterilization may be automatic, semi-automatic, or may require some action on the part of the user. Self-sterilization including re-sterilization (in most instances a skin piercer will be provided sterilized) may occur in any suitable manner. In certain embodiments, the automatic or semi-automatic self sterilizing mechanism may be activated, e.g., automatically, just prior to the initiation of a biological fluid test. Such sterilization may be computer implemented such that the methods of sterilization may be computer-implemented methods of sterilization.

For example, the self sterilization function might be activated when the skin piercing member holder 130 is cocked for use. Self sterilization might take as little as a few seconds. In other embodiments, the self sterilization function may be activated substantially immediately after completion of a biological fluid test. For example, after a skin piercing member has retracted back into the skin piercing member holder 130, or upon repositioning of a cover over the skin piercing member(s), the skin piercer may be sterilized. In embodiments that include covers over at least part of the skin piercing member, sterilization may occur while the skin piercing member is either partially or wholly within the cover, other times it may occur prior to covering with a cover. In certain embodiments the method of sterilization may be partially or fully contained within the cover. In certain embodiments the skin piercing member(s) 140 may be automatically or semi-automatically sterilized while in the skin piercing member holder 130.

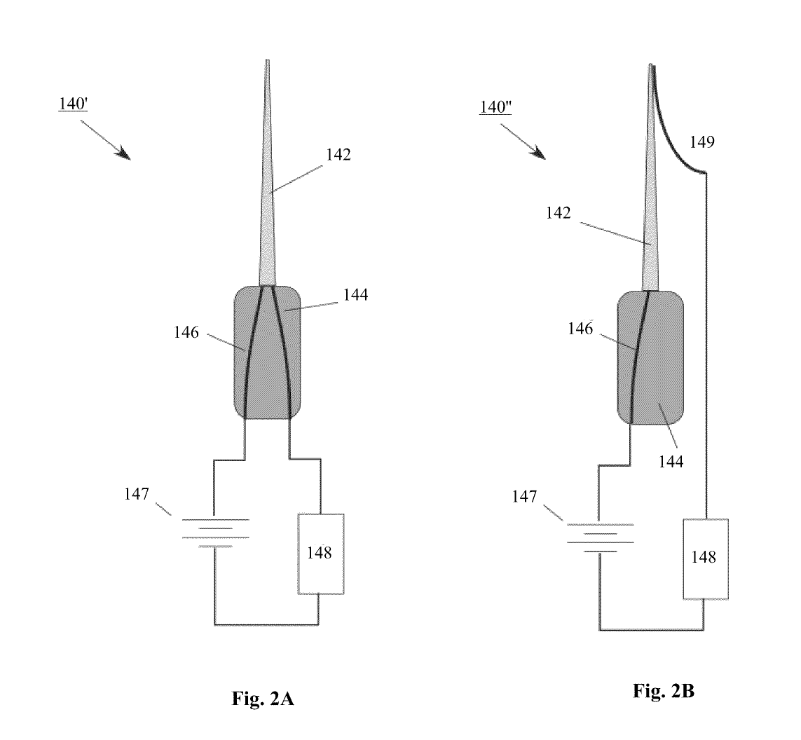

Any suitable sterilization process may be used. FIGS. 2A and 2B shows embodiments of a device and method of sterilization of a skin piercing member. The embodiment of FIG. 2A shows skin piercing member 140′ having lance 142 connected to body 144, which includes leads 146 connected to the part of the skin piercing member that pierces the skin and to power supply 147. Any suitable manner of supplying power may be employed. For example, a battery and/or a switching device 148, a manual switch, or a switch operated by a microcontroller. In such embodiments, sterilization is accomplished by passing a pulse of current through tip 142 of the skin piercing member to generate resistive heating. A short pulse of current is provided to leads 146. High electrical resistance in the tip material causes it to heat to a temperature that is sufficient for sterilization. For example, 200° C. for a period of time, e.g., from less than about 1 second to about 60 seconds, e.g., from less than about 10 seconds, e.g., less than about one second. The tip may then cool by free convection to ambient temperature which may take as little as about a few seconds. Any suitable materials may be used for the various components of the self sterilizing skin piercing member. For example, in FIG. 2A, body 144 may be ceramic, but any suitable insulating material may be employed. The current source may be integrated into interior space 111 of the device. Any suitable configuration of circuitry for delivering the electrical pulse may be employed.

FIG. 2B shows an embodiment of the self sterilizing skin piercing member 140″ that employs at least one lead 149 contacting the tip of the skin piercing member from outside the skin piercing member, e.g., as a brush contact. This may provide a more uniform current distribution within the tip of the skin piercing member and thus more uniform heating of the tip of the skin piercing member. A brush-type lead is an electrical lead or contact which makes physical contact but which is not permanently attached. With one lead contacting the base of the skin piercing member and another brush-type lead contacting near the sharp end of the skin piercing member, current may flow more completely and uniformly throughout the skin piercing member. Correspondingly, thus resistive heating may occur more completely and uniformly throughout the skin piercing member.

In further aspects, embodiments of the present disclosure include lancing devices that contain multiple skin piercing members which are linked together within the interior space of a housing. In some embodiments, the lancing device may be used as a stand-alone device, e.g., as a separate part of an analyte testing kit which includes a meter, one or more test strips and a lancing device as described herein. In some embodiments the internal mechanism of the subject disclosure may be partially or fully incorporated to be integrated with or coupled to one or more additional devices used for analyte testing. For example, embodiments of lancing devices of the subject disclosure may be coupled or coupleable (e.g., post-manufacture such as by a user) to an analyte test meter, for example the analyte test meter having test strip disk with having one or more test strips as described above in conjunction with FIG. 1.

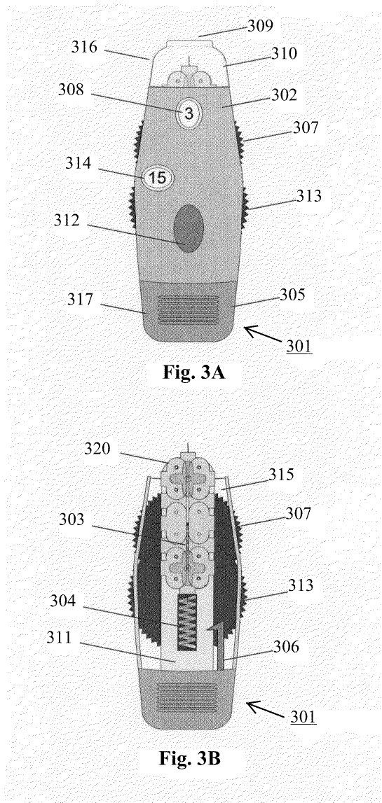

Turning now to FIGS. 3A and 3B, the figures show an embodiment of the subject lancing device that is configured to retain multiple lancets at the same time within an interior space of the lancet device housing. In this particular embodiment, the lancets are linked together as a chain to position and move the lancets within and in and out of the interior space. The lancing devices may be configured to hold any number of lancets, for example as few as one or as many as 100 or more.

Lancet device 301 includes housing 302 having an interior space 315. Interior space 315 is dimensioned to accommodate a plurality of lancets at one time, as described in greater detail herein. In some embodiments the exterior housing has a housing geometry configured for ease of use, even by users suffering from impaired dexterity or eyesight. Housing 302 includes the overall outer envelope of lancing device 301 and, in an example embodiment, includes an endcap 310 and front and back housing shells, or may otherwise include access to the interior of the housing so a user may replace interior components such as the plurality of lancets. In certain embodiments, the overall length L of housing 302 is less than about 10 times, e.g., less than about 4 times, e.g., less than about 3 times, the overall width W. This aspect ratio (i.e., L/W) of no more than 10:1, 4:1, 3:1, provides the lancing device 301 with improved stability in use, which is of particular benefit in enabling users with impaired dexterity to apply pressure between the forward end of the device and a sampling site. In other embodiments, the ratio of width to height may be greater than 10:1, for example 15:1, or less than 3:1, such as 1:1.

The forward end 316 may include a forward panel oriented generally perpendicular to the stroke of a lancet carried by the lancing device. Accordingly, in such embodiments pressure may easily be applied for sufficient sampling, between the forward end 316 and a sampling site, by applying compressive force to the rear end 317 in an axial direction, while maintaining the device in a stable and upright position, see., e.g., U.S. Pat. No. 6,283,982, the disclosure of which is herein incorporated by reference. In such embodiments, the stroke of the lancet is oriented generally perpendicular to the skin at the sampling site, to provide a straight in-straight out lancing of the skin for improved user comfort.

Device 301 includes movable carriage 311 that couples to the lancets 324, and more specifically, the lancet roller chain assembly 320. Also provided are firing spring 303, recoil dampening spring 304 (springs 303 and 304 may be a single spring or may be two or more as shown herein), arming mechanism 5, optional lockable firing mechanism 306, depth adjustment mechanism 307, depth adjustment indicator 308, lancet advancement mechanism 313, lancet indicator 314, removable endcap 310, housing opening 309, e.g., blood expression feature, and firing button 312.

The lancet carriage 311 may be translationally mounted to slide within the housing for carrying lancet roller assembly 320 so that a lancet of assembly 320 is carried along a reciprocating stroke from a retracted position wherein the lancet is substantially entirely within the housing, through a cocked position, to an extended position wherein at least a portion of the lancet extends through the opening of the housing a distance outwardly beyond the housing, and back to the retracted position.

For example, an embodiment includes an assembly coupled to a frame and including a plurality of lancets linked together as a chain of lancets. The assembly is coupled to the frame so that it is moveable about the frame as a single unit to position a lancet with respect to the opening, e.g., aligned or alignable with the opening.

A moveable carriage coupled to the assembly is configured to move a lancet of the assembly that is positioned with respect to the opening along a stroke from position wherein the lancet is substantially entirely within the interior space to an extended position wherein at least a portion of the lancet extends a distance outwardly through the opening, and back to a position within the interior space.

The cocking or arming mechanism 305 shifts the lancet carrier from the retracted position to the cocked position, and driving mechanism drives the lancet from the cocked position to the extended position, and a retraction mechanism, returns the carriage from the extended position to the retracted position. In this particular embodiment, the driving mechanism for driving a lancet of the lancet roller chain assembly from the cocked position to the extended position includes a compression spring 303. Spring 303 biases the carriage 311 toward its extended position, and serves to propel the carriage along a tissue-penetration portion of its stroke. In certain embodiment, carriage 311 may be configured to be constrained to slide generally linearly parallel to the central axis of the housing. For example, carriage 311 may be configured to provide a generally linear stroke along or adjacent approximately the central axis of the housing so that a lancet is driven along a substantially straight in-straight out path, penetrating the skin of the sampling site at an angle generally perpendicular to the skin surface.

Cocking mechanism arms the lancing device by placing the firing spring 303 into compression, storing potential energy which is converted to kinetic energy as the lancet is driven through the tissue-penetrating portion of its stroke. The cocking mechanism may be actuated by the user by grasping an arming actuator 305 adjacent the rear end 317 and pulling cocking actuator 305 away from the housing. Cocking actuator 303 may include first and second halves that are attached to opposite sides of a cocking carriage (not shown), whereby retraction of the cocking carriage draws carriage 311 rearward into a cocked position in which spring 303 is compressed. As carriage 303 is drawn into its cocked position, the tip of lockable firing mechanism 306 retains carriage 311 in its cocked position until released by the triggering mechanism.

Recoil or return spring 304 draws the arming mechanism 305 back against the housing after the lancing device is cocked. Spring 304 may also serve to retract carriage 311 from its extended position to its retracted position after triggering the device, thereby withdrawing a lancet from the tissue of the sampling site. Spring 303 may be relatively stiffer than spring 304 in certain embodiments, so that upon triggering the device, spring 303 drives carriage 311 through its entire stroke, from the cocked position to the extended position, before spring 304 withdraws the carriage.

The lancing device also includes a triggering mechanism for releasing carriage 311 from its cocked position. A trigger or firing button 312 may be mounted on the front of housing 302. When the user presses button 312, the lockable firing mechanism 306 disengages to release carriage 311, which is then driven by spring 303 through the tissue-penetrating portion of its stroke to the extended position, whereupon return spring 304 then retracts the carriage 311 to its retracted position.

The lancing device may include an adjuster, e.g., a stroke control or depth adjustment mechanism, for selectively controlling the stroke range, and thereby controlling the depth of penetration of a lancet into the body tissue of the sampling site. The stroke control mechanism may limit the range of the stroke of carriage 311 in the forward direction, and define the extended thereof. In certain embodiments, the stroke control mechanism is adjustable to permit the user to selectively adjust the stroke range and thereby varying the depth of penetration of a lancet into the body tissue of the sampling site. A stroke adjustment mechanism may include a thumbwheel. Depth adjustment thumbwheel 307 is rotatably mounted to housing 302. As used herein, the term “thumbwheel” is non limiting and includes rotating dials or other elements, translating slide mechanisms, or other movable stop mechanisms, that are manipulable in any way by the user, and specifically is not limited to elements manipulable by the thumb of a user or by any particular body part of the user.

The lancing device may also include a depth indicator 308 for indicating the set stroke range and, correspondingly, the depth of penetration of a lancet into the skin. According to certain embodiments, depth indicator 308 includes indicia on the thumbwheel 307, and a cooperating opening or window through the housing for displaying the indicia. The stroke adjustment mechanism of the lancing device may further include indexing means to allow the user to increment the forward extent of the stroke range, and accordingly the penetration depth, through a plurality of discrete positions.

Lancing device 301 may also include a pressure applicator for tensioning skin at the sample site through the application of compressive pressure against the sample site, and for stimulating the generation of a sample of body fluid of a desired quantity. In certain embodiments, the pressure applicator includes the forward end of endcap 310 that is releasably attached to the forward end of the housing. Endcap 310 is shown attached in FIG. 3A. An opening 309 is defined for allowing passage of at least a portion of a lancet, and in certain embodiments may allow passage of a portion of lancet roller chain assembly 320.

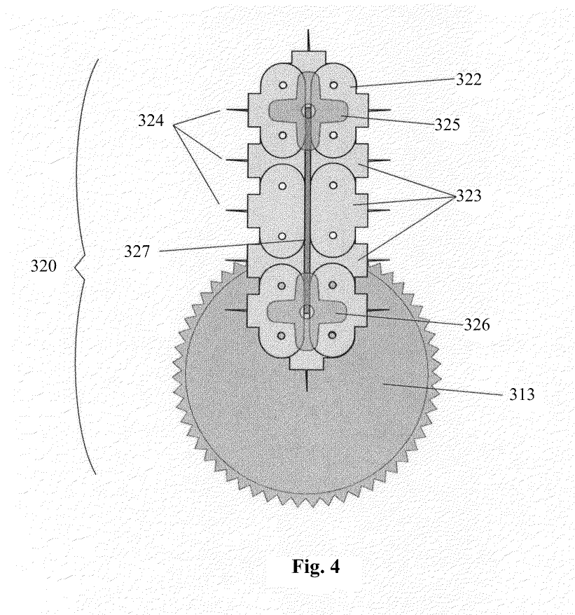

FIG. 4 shows a cut-away view of lancing device 301 with some elements removed. As described above, the subject lancing devices include a plurality of lancets in an interior space of a housing. Device 301 includes lancet linkage assembly 320 attached to spring loaded carriage 311. Assembly 320 includes a plurality of lancets 324 linked together. The individual lancets are arranged to be positioned on or about the links 323 of a roller chain 322. As shown, each individual link of the roller chain assembly contains a single lancet. Description of a roller chain is exemplary only. It is understood that any suitable lancet linking member may be employed, e.g., a belt may be used rater than a roller chain. Positioning individual lancets on the links of a roller chain (or other linking member) provides a very compact way of storing a large number of lancets in a lancing device while providing quick, safe and simple access to a fresh lancet when needed.

In certain embodiments the roller chain 322 is wrapped around at least one sprocket, e.g., a front 325 and rear 326 sprocket, e.g., similar to a chain on a bicycle. Sprockets 325 and 326 are rotatably attached to a frame 327 which fixes their orientation and spacing. In some embodiments one or both sprockets 325 and 326 may rotate around their axis as to advance or move roller chain 322. Advancement of the roller chain assembly may be manual, semi-automatic or fully automatic. In embodiments that are semi or fully automatic, the advancement function may be activated at any time. For example, it may be initiated when cocked for use or it may advance substantially immediately after completion of a given lancing event and/or an analyte test. For example, after a lancet has retracted back into the lancing device, or upon repositioning of a cover over the lancet sharp(s) 324, the lancet assembly may advance to move an adjacent, un-used lancet into position for use. Any mechanism of advancing the roller chain assembly may be used. For example, advancement of the roller chain assembly may include one or more fingerwheels, buttons, slide switches, track balls, dials, knobs, joysticks, etc. For example, lancet advancement mechanism herein in the form of thumbwheel 313 may be used to move assembly 320. Thumbwheel 313 is rotatably mounted to housing 302. As used herein, the term “thumbwheel” is non limiting and includes rotating dials or other elements, translating slide mechanisms, or other movable stop mechanisms, that are manipulable in any way by the user, and specifically is not limited to elements manipulable by the thumb of a user or by any particular body part of the user.

The lancing device may also include lancet indicator 314 for indicating a lancet number. The number may correspond to how many unused lancets still remain in the lancing device, how many lancets have been used since the last reset, etc. Lancet indicator 314 may be manual, automatic or semi-automatic. For example, indicator 314 may be configured to reset automatically every time the lancet(s) or lancet assembly is replaced or may necessitate that the user manually reset the indicator using any suitable means, i.e., thumbwheel, button, knob, switch, or the like. In a similar manner, in some embodiments the lancet indicator may advance or reset automatically every time the lancet assembly is advanced or it may be manually adjusted by the user. According to certain embodiments, lancet indicator 314 includes indicia on the thumbwheel 313, and a cooperating opening or window through the housing for displaying the indicia. Lancet advancement mechanism 313 of the lancing device may further include indexing means to allow the user to increment the advancement of assembly 320 through a plurality of discrete positions.

In certain embodiments, the lancing devices may include a mechanism to prevent a user from indexing the lancet linkage assembly 320 backwards to a previously used lancet, e.g., such as a ratchet and pawl mechanism or the like, so as to allow indexing of the lancet linkage assembly in only one direction. Additionally, some embodiments may have a mechanism, e.g., an advancement stop, to cease to advance assembly 320 until the lancet(s) or lancet assembly has been replaced with unused lancet(s). A stop may be a protrusion or the like positioned on the housing and/or assembly 320, or elsewhere. In some embodiments, a mechanism may be included that advances (or requires to be advanced) lancet linkage assembly 320 to an unused lancet after each firing of the lancet device.

Lancet linkage assembly 320 may be partially or wholly replaceable, e.g., after all lancets have been used. In some embodiments the entire lancet assembly may be removed, discarded, and replaced with a new lancet assembly that includes lancets. In certain embodiments, only the lancet(s) of lancet linkage assembly 320 may be individually replaceable. Another embodiment may allow a user both the option to replace lancet linkage assembly 320 as well as the option to replace just individual lancets without need to replace lancet linkage assembly 320. For example, frame 327 and/or sprockets and/or lancets may be disposable and replaceable or may be reusable. In yet another embodiment, the entire lancing device with the lancet assembly may be fully disposable.

Certain embodiments may include two or more separate portions of the housing. The separate portions of the housing may have complementary, interlocking structures, such as, for example, interlocking ridges or a ridge on one component and a complementary groove on another component, so that the two or more separate components may be easily and/or firmly coupled together. This may be useful, particularly if the components are taken apart and fit together occasionally, for example, if/when replacement of lancet linkage assembly 320 or individual lancets 324 is necessary. However, other fasteners may also be used to couple the two or more components together, including, for example, screws, nuts and bolts, nails, staples, rivets, or the like. In addition, adhesives, both permanent or temporary, may be used including, for example, contact adhesives, pressure sensitive adhesives, glues, epoxies, adhesive resins, and the like.

Embodiments may include covers at least over the sharp portion of the lancet(s). Covers may be automatically moved or removed by the device or may be pierceable by the lancet so that removal is not necessary. In certain embodiments, covers may include a cover removing and/or replacing mechanism contained within the interior space of the housing that maintains a removed cover within the housing. For example, a cover removing and/or replacing mechanism may remove a cover from a lancet and maintain it within the housing, the lancet may be used to pierce, and the cover may be replaced on the used lancet. In other embodiments, a cover may be removed from a lancet and ejected from the device. Covers for lancets that may be employed in certain embodiments include, but are not limited to, those described in U.S. patent application Ser. No. 11/396,355, the disclosure of which is herein incorporated by reference.

In certain embodiments, the lancets may be ejected or ejectable from the device after use, or may retract or may be retractable into device after use wherein at least the sharp portion of the lancet will not be exposed to the user. Accordingly, certain embodiments may be configured to allow a user to discard the entire lancet assembly with the lancets retracted while other embodiments may include a mechanism to eject just the lancets and replace them individually.

In certain embodiments, lancing device 301, or certain components thereof, may be integrated with an analyte testing system. Such an integrated system may include, together in a single housing, components of lancing device 301, one or more analyte test strips (e.g., glucose test strip), and an analyte meter to determine analyte results of a sample applied to a test strip. For example, housing 302 of lancing device 301 may include one or more analyte test strips and an analyte meter. The strips may be coupled to assembly 320 or otherwise.

In certain embodiments, the lancing device 301 may be integrated with the analyte testing device having a test strip disk having one or more test strips as described above in conjunction with FIG. 1. Other exemplary systems which may be used with the lancing devices herein, including integrated or otherwise coupled thereto, include but are not limited to U.S. application Ser. Nos. 11/535,985, 11/535,986, 12/233,584, 61/099,184, 61/102,640, the disclosure of each of which are incorporated herein by reference for all purposes.

Referring again to FIG. 1, one or more microprocessors with a power supply may be coupled to housing 110, including being fully contained therein, and may include programming to control aspects of the device. For example, one or more microprocessors may carry programming instructions to control the mechanical movements of the device and/or the timing of the testing and/or the skin piercing member firing. One or more microprocessors may read a test strip and determine an aspect of an analyte, e.g., presence and/or concentration of glucose, in the sample applied to the test strip. One or more microprocessors may conduct the analyte test, and may also store the analyte data, such that interior space 111 may include analyte meter suitable componentry, e.g., circuitry. A meter may be included in the housing and connected or connectable to one or more of the test strips include but are not limited to meters as described in U.S. Pat. Nos. 6,071,391; 6,120,676; 6,143,164; 6,299,757; 6,338,790; 6,377,894; 6,600,997; 6,773,671; 6,377,894; 6,600,997; 6,773,671; 6,514,460; 6,893,545; 6,924,518, the disclosures of which are herein incorporated by reference for all purposes.

Certain embodiments include a timing circuit that may be actuated by one or more microprocessors of the device with a timing function executed by software. The timing circuit may be programmed or programmable to a determined a timing interval to test for analyte according to one or more schedules. The timing of analyte testing (clock period) may be predetermined and programmed, e.g., about once every 4-6 hours or the like, so that a test is thusly performed according to the schedule automatically or at least semi-automatically (for example, prior to initiation of a test a user may be required to confirm that a test should be performed). Timing may be computer implemented such that the processes of piercing the skin and testing analyte at given times may be computer-implemented processing.

Testing schedules may be programmed during manufacture or may be programmed post-manufacturing, e.g., by a user and/or healthcare provider. However, in alternative embodiments the clock period may be adjustable such as automatically adjustable, (may be dynamic), e.g., based on how often measurements are needed, learned behavior of activity of a user, or the like, so that information from previous tests provides information upon which to modify one or more testing schedules. The one or more microprocessors may store such data (inputted by a user and/or collected automatically by the device), contemplate the data in a dynamic clock period algorithm, and determine and execute- or at least recommend to a user via a reporting module—a modified clock period based on the results of the algorithm. The device's ability to automatically modify testing aspects according to learned behavior is particularly useful to customize a given device for a given user and to maintain tight glycemic control. For example, a device may be configured to “learn” eating and/or exercising schedules and/or periods of illness or stress, etc., of a user and may automatically (without any user intervention) modify testing schedules accordingly. In some embodiments, a device will not execute a modified schedule without confirmation from a user. For example, a device may determine a modified schedule, report it to a user via a reporting module prior to implementation, and confirmation by a user will be required to confirm or reject initiation of the modified scheduled by some affirmative action. Confirmation to initiate a test may be in the form of a selector, e.g., in the form of a menu displayed on a screen that is accessed by the user or another individual by using buttons on the surface of device 102 or other connected device. In other embodiments, a dial selector, dedicated buttons, a touch screen, voice command, or the like, may be used. If confirmed, the one or more microprocessors of the device will execute the current schedule instead of the modified schedule. Accordingly, a modified analyte testing timing sequence may be easily incorporated and executed by device 102. As noted above, adjustability may be automatic or semi-automatic such that the device may be an “intelligent” device, or may be re-programmed by a user or health care provider or the like. In certain embodiments, a clock may run faster or slower triggering measurements more or less frequently depending on how quickly the blood glucose level can change, or the like. The modified clock speeds may be accomplished automatically or semi-automatically, e.g., may require confirmation.

The one or more microprocessors employed in embodiments of the subject disclosure may be any suitable processor(s). In certain embodiments, one or more may be a digital integrated circuit, including an application specific integrated circuit (ASIC). Accordingly, one or more microprocessors of device 102 may serve one or more of the following control functions: 1) timing for specific tasks or for the entire device; 2) together with program and data memory, storing data corresponding to the analyte measured at specified time intervals; 3) calculating analyte levels from the stored data; 4) outputting analyte information concentration data to a reporting module of device 102 and/or of another device; 5) transmitting raw or processed (e.g., filtered, digitized, etc.) data to another device, and the like. Memory may be a digital integrated circuit which stores data and the microprocessor operating program. A reporting module may take any one or more of various hard copy and/or soft copy forms. For example, it may be a visual display, such as a liquid crystal (LCD) or LED display, a tape printer, audible signal, or the like, or any combination, and may be located on device 102 or elsewhere.

Device 102 also includes a power source, e.g., to power the movement of holder 130 and to arm or energize a skin piercing member, etc. As shown in FIG. 1, a drive spring 120 may be integrated into interior space 111 and may provide power for one or more tasks of the device. In certain embodiments, the drive spring 120 may also provide power to the one or more microprocessors. FIG. 1 shows rotating timing mechanism, rotating firing mechanism, and one or more microprocessors—designated herein collectively as reference numeral 132 for exemplary purposes, as part of rotatable holder 130, but each one or more of these components could be positioned elsewhere in interior space 111.

Also included is a firing mechanism to fire a skin piercing member into the skin, and more specifically to advance the skin piercing member through the opening of disk 114 to pierce the skin of the user. Any suitable firing mechanism may be employed. For example, various types of springs such as torsion springs, compression springs or extension springs may be used.

As noted above, housing 110 may include a reporting module (not shown) to report analyte results to a user. Reporting modules include those described herein and others. However, the housing may be devoid of a reporting module. In such embodiments, the housing may include a transmitter to transmit analyte test results to a remote receiver that includes a reporting module such as a display. In this way, discrete testing may be even more fully realized. In some embodiments, the housing may be substantially free—including completely free—of user interface components such as displays, buttons, etc., so that the housing may be small and extremely low in profile.

A receiver unit for receiving analyte information from device 102, if employed, may be any suitable receiver and may be any suitable size and shape. In certain embodiments, a receiver unit is a computing device. A receiving device may be a small, hand held device such as about the size of a small cell phone or pager, or in many embodiments may be integrated into a cell phone or pager, and/or may be a desktop or laptop computer. In certain embodiments, a receiver may be integrated into an automobile so that analyte data may be transmitted via a wired or wireless connection to a reporting module of an automobile such as a display module, audio system, etc.). Results of analyte test may be transmitted to a receiver via any suitable process of transmission. For example, transmission may be achieved by using Bluetooth® technology, radio frequency (RF) transmission signal, or by using optical or wired connection/cable. In one embodiment, analyte test data may be stored in either the integrated analyte test device 102 or in the receiver unit, or both.

In certain embodiments the housing may include a reporting module in the form of a display to display all or partial results to a user in any suitable visual form such as, numeric, graphical, iconic, etc., or any combination thereof. The size of the display may be any suitable size. In certain embodiments the display is selectable so that a user may be able to select various display fonts for ease of use. When a larger font is selected, the display may employ scrolling text or a button may be used to advance through text. The housing may include no buttons, or may include one or more buttons or very few, e.g., to turn on the device and/or confirm modified testing schedules if such function is included. One or more of the buttons may have multiple functionalities. For example, one or more buttons may turn the device on/off, they may be used to begin/end analyte testing, they may control the display, and they may be used to activate the transmit feature, or may control any one or combination of functions. In certain embodiments, the data transmission may be activated automatically or semi-automatically. Transmission may initiate automatically or may be initiated semi-automatically upon completion of each analyte test or may be initiated at any time thereafter via actuation of a transmit actuator on either the transmitting integrated device or on the receiver unit.

Certain embodiments include devices that include programming to determine a drug delivery dose based on a determined analyte level, e.g., insulin amount to be delivered to a user based on glucose level determined by one or more glucose test strips of the device. In some instances, the device may include or be coupled to a drug delivery module having a reservoir of drug integrated in the interior space of the housing of the device. An amount of drug may be determined (e.g., basal amount, bolus amount, or the like) by one or more microprocessors programmed therefore, and delivered automatically, or may be determined and reported to a user for confirmation or rejection or modification prior to delivery. If confirmed (or modified), the device may execute the delivery function. Delivery may be by way of a soft cannula or needle coupled to the drug reservoir and insertable in a user. The device may include data input so that a user may enter additional information into the device such as carbohydrate data, exercise data, etc., that may be contemplated by the drug delivery determination algorithm.

The test strips of the subject disclosure may be adapted to determine a wide variety of analytes, where glucose is primarily used herein for exemplary purposes only and is in no way intended to limit the scope of the disclosure. Additional analytes include, for example, acetyl choline, amylase, bilirubin, cholesterol, chorionic gonadotropin, creatine kinase (e.g., CK-MB), creatine, DNA, fructosamine, glucose, glutamine, growth hormones, hormones, ketones, lactate, peroxide, prostate-specific antigen, prothrombin, RNA, thyroid stimulating hormone, and troponin. The concentration of drugs, such as, for example, antibiotics (e.g., gentamicin, vancomycin, and the like), digitoxin, digoxin, drugs of abuse, theophylline, and warfarin, may also be determined. A given device may be adapted to determine more than one analyte, e.g., glucose and ketones, or the like.

Any suitable biological fluid may be tested according to embodiments herein, and include, but is not limited to, any body fluid in which the analyte can be detected, for example, blood (which includes whole blood and its cell-free components, such as, plasma and serum), interstitial fluid, dermal fluid, sweat, tears, urine and saliva.

The test strips of the subject disclosure are described primarily as electrochemical sensors for exemplary purposes only, where such description is in no way intended to limit the scope of the disclosure. It is to be understood that the test strips may be other than electrochemical test strips, e.g., optical test strips, etc.

The analyte test strips of the present disclosure may be adapted to measure the concentration of an analyte in any volume of sample, but are particularly useful in the determination of analyte concentration in a small volume of sample, e.g., a sample having a volume no more than about 1 μL, for example no more than about 0.5 μL, for example no more than about 0.25 μL, for example no more than about 0.1 μL. In some embodiments, the volume of sample may be as low as 0.05 μL or as low as 0.03 μL. The test strips of the subject disclosure may be configured as those described herein and, e.g., in U.S. Pat. Nos. 6,071,391; 6,281,006; 6,103,033; 6,338,790; 6,120,676; 6,143,164; 6,616,819; and U.S. application Ser. Nos. 11/282,011; 11/225,659; 11/237,447; 11/277,931; 11/535,986, the disclosures of which are herein incorporated by reference for all purposes. Integrated systems with which embodiments of the subject disclosure may find use include, e.g., U.S. application Ser. Nos. 11/535,985; 11/585,986, 12/233,584, 61/099,184, 61/102,640, the disclosures of which are herein incorporated by reference for all purposes.

Sensors are generally configured for use with an electrical meter, which may be connectable to a PC or other electronics. The connection may be wired or wireless. Exemplary test strips are described in greater detail herein, where test strips used in the subject disclosure may include some or all of the features and components described.

Test strips generally may have first and second substrates which are each non-conducting, inert substrates and which form the overall shape and size of the sensor. Substrates may be substantially rigid or substantially flexible. In certain embodiments, substrates are flexible or deformable. Examples of suitable materials for substrates include, but are not limited, to polyester, polyethylene, polycarbonate, polypropylene, nylon, and other “plastics” or polymers, and the like. In certain embodiments the substrate material is “Melinex” polyester. Other non-conducting materials may also be used such as paper, etc.

Positioned between the substrates may be a spacer to separate the substrates and to provide a sample chamber, e.g., a cut-out in a spacer may form a sample chamber of a test strip. A spacer is usually an inert non-conducting layer, and generally may be at least as flexible and deformable (or as rigid) as the substrates. In certain embodiments, a spacer is an adhesive layer or double-sided adhesive tape or film. Any adhesive selected for spacer should be selected to prevent or minimize diffusion or the release of material which may interfere with accurate analyte measurement. The thickness of a spacer defines the volume of the sample chamber and may be dimensioned to provide a sample chamber having a capillary volume. The length of a spacer may be less or greater than the length of one or both of the substrates. The width of a spacer may be the same or different than the widths of one or both of the substrates. The shape of a test strip may be any suitable shape. In many embodiments, a test strip is in the form of a rectangular strip. However, other shapes, including complex shapes, are envisioned as well.

As described above, the test strips include a sample chamber for receiving a volume of biological fluid sample to be analyzed, which chamber includes one or more sample chamber entrances, e.g., positioned about one or more intersecting edges of the sensor. A sample chamber is configured so that when a sample is provided in a sample chamber, the sample is in electrolytic contact with both the working electrode and the counter electrode, which allows electrical current to flow between the electrodes to effect the electrolysis (electrooxidation or electroreduction) of the analyte. A sample chamber has a volume sufficient to receive a sample of biological fluid therein. In some embodiments, such as when a test strip is a small volume sensor, a sample chamber has a volume that is no more than about 1 μL, for example no more than about 0.5 μL, and also for example, no more than about 0.25 μL, e.g., a volume of no more than about 0.1 μL, e.g., no more than about 0.05 μL, e.g., no more than about 0.03 μL. A sample chamber has dimensions that facilitate drawing sample to be analyzed into the sample chamber by capillary or other surface tensions forces. A test strip includes a working electrode and at least one counter electrode. The counter electrode may be a counter/reference electrode. If multiple counter electrodes are present, one of the counter electrodes will be a counter electrode and one or more may be reference electrodes. An indicator electrode may also be provided to indicate if/when a sample chamber is full and/or has sufficient sample to perform an analyte test.

At least one working electrode is positioned on one of the substrates of a test strip. Certain embodiments may include two or more working electrodes. A working electrode has a portion present in the area of a sample chamber, and in some embodiments, includes a conductive trace that extends from the sample chamber to an end of the sensor, such as for connecting to a meter. A working electrode may be a layer of conductive material such as any suitable conductive material, e.g., gold, carbon, platinum, ruthenium dioxide, palladium, or other non-corroding, conducting material. A working electrode may be a combination of two or more conductive materials. An example of a suitable conductive epoxy is ECCOCOAT CT5079-3 Carbon-Filled Conductive Epoxy Coating (available from W.R. Grace Company, Woburn, Mass.). The material of working electrode may have a relatively low electrical resistance and is typically electrochemically inert over the potential range of the sensor during operation.

A working electrode may be applied on a substrate by any of various processes, including but not limited to, vapor deposition or vacuum deposition or otherwise sputtered, printed on a flat surface or in an embossed or otherwise recessed surface, transferred from a separate carrier or liner, etched, or molded, printed, including but are not limited to, screen-printing, piezoelectric printing, ink jet printing, laser printing, photolithography, and painting.

As described above, at least a portion of a working electrode is provided in sample chamber for the analysis of analyte, in conjunction with the counter electrode. A test strip includes at least one counter electrode (or counter/reference) positioned within the sample chamber. A counter electrode may be applied to a different substrate than that of the working electrode to provide a sensor having oppositely-oriented, spaced-apart electrodes, or a counter electrode may be applied to the same substrate as the working electrode to provide a sensor having co-planar electrodes. A counter electrode also has a portion present in the area of sample chamber, and in some embodiments, includes a conductive trace that extends from the sample chamber portion to an end of the test strip, such as for connecting to a meter.

A counter electrode may be constructed in a manner similar to a working electrode. The same materials and processes may be used to make counter electrode as are available for constructing working electrode, although the same or different materials and processes may also be used. A counter electrode may include a mix of multiple conducting materials, such as Ag/AgCl and carbon.

A test strip may include at least one indicator, such as an indicator electrode positioned on one or both of the substrates. An indicator electrode is used to detect when sample chamber has been completely and/or sufficiently filled with sample, to prevent partial filling of the sample chamber in those embodiments in which complete filling is required. In some instances, less than complete filling of a sample chamber, but a minimum amount, is acceptable to perform an accurate analyte test. Upon the sample contacting the indicator electrode, the indicator electrode may be the source of a signal to an attached meter. Suitable signals include, for example, voltage, current, resistance, impedance, or capacitance. The signal indicates to the meter, and/or the user, that there is sufficient sample in the sample chamber to begin the assay. This indication may be a visual sign and/or auditory signal and/or vibratory signal, or the meter may be configured to automatically initiate the assay.

An indicator electrode may be constructed in a manner similar to a working electrode and/or a counter electrode. Suitable materials and processes for an indicator electrode include the same materials and processes as used for a working electrode and/or a counter electrode, although different materials and processes may also be used. Carbon is a material that may be used for an indicator electrode.

In certain embodiments, an indicator electrode may be positioned in a sample chamber with at least one working electrode positioned between it and an entrance to the sample chamber. In some embodiments, a counter electrode will also be positioned between the indicator electrode and the chamber entrance. An indicator electrode may be positioned so that that biological fluid sample, upon entering a sample chamber via chamber entrance, flows past a working electrode prior to contacting the indicator electrode.

Chemistry (also may be referred to as sensing chemistry, analyte-responsive chemistry or reagent(s), and the like) is provided for the analysis of the analyte. This sensing chemistry may include a redox mediator and a second electron transfer mediator, although in some instances, one or the other may be used alone. The redox mediator may be air oxidizable. Placement of chemistry components may depend on a variety of factors. For example, component(s) may form a sensing layer on one or more working electrodes. Alternatively, one or more components may be present on any surface in a sample chamber prior to the introduction of the sample to be analyzed. As another example, one or more component(s) may be placed in the sample prior to introduction of the sample into the sample chamber.

A redox mediator may be disposed on at least one or more working electrode as, for example, a layer. In an embodiment having a redox mediator and a second electron transfer agent, then both components may be disposed on a working electrode as individual layers, or combined and applied as a single layer. A redox mediator enables the electrochemical analysis of molecules which may not be suited for direct electrochemical reaction on an electrode. A mediator functions as an electron transfer agent between the electrode and the analyte.

Any suitable chemistry may be employed in the test strips of the subject disclosure. For example, a redox mediator that may be employed is a transition metal compound or complex. Examples of suitable transition metal compounds or complexes include but are not limited to osmium, ruthenium, iron, and cobalt compounds or complexes. In these complexes, the transition metal is coordinatively bound to one or more ligands, which are typically mono-, di-, tri-, or tetradentate. The redox mediator can be a polymeric redox mediator, or, a redox polymer (i.e., a polymer having one or more redox species). Examples of suitable redox mediators and redox polymer are disclosed, e.g., in U.S. Pat. Nos. 6,338,790, 6,605,200 and 6,605,201, the disclosures of which are herein incorporated by reference.

A test strip may include a redox mediator and a second electron transfer agent that is capable of transferring electrons to or from the redox mediator and the analyte. One example of a suitable second electron transfer agent is an enzyme which catalyzes a reaction of the analyte. For example, a glucose oxidase or glucose dehydrogenase, such as pyrroloquinoline quinone glucose dehydrogenase (PQQ), is used when the analyte is glucose. Other enzymes may be used for other analytes. These enzymes catalyze the electrolysis of an analyte by transferring electrons between the analyte and the electrode via the redox mediator.

Test strip embodiments described herein include those that are sandwiched or layered constructions having substrates spaced apart, such as by spacer. Such a construction may be made by laminating the various layers together in any suitable manner, or made using any suitable processes. Test strips may also be molded.

Molding may include positioning at least two spaced apart, electrically conductive electrodes (e.g., wires) in a mold, and molding a body of insulative material around the electrodes, so that at least one sample chamber opening is provided about a corner of the sensor. More specifically, molding may include positioning at least two spaced apart, electrically conductive electrodes (e.g., wires) in a mold, before or after molding, treating at least one of the electrodes with one or more chemicals to change the electrical properties of the treated electrode upon contact with a fluid sample, and molding a body of insulative material around the electrodes with one end having at least one sample chamber opening is provided about a corner of the sensor. The body may be molded in multiple pieces, e.g., two pieces, with a body and end cap for attaching to one another after the molding is completed, or in a single piece.

Also provided are processes of analyte determination, including processes of analyte testing that includes testing with more than one test strip without displacement of the device from a given location on a body of a user. In general, embodiments include positioning an analyte testing device that includes at least one test strip and at least one skin piercer (in many embodiments at least two test strips) on a skin surface of a user and holding it in place (fixed in place) for a period of time, e.g. from about 1 hour to about 1 month; cocking a skin piercer of the device and firing the cocked skin piercer to pierce the skin, expressing biological fluid from the pierced site, contacting the expressed sample with a test strip of the device, and determining an aspect of the analyte, e.g., presence and/or concentration of the analyte. Embodiments include repeating the above sequence one or more times, including automatically executing a sequence according to a timing schedule. As such, the sequence of actions may be repeated one or more times so that multiple tests may be performed with multiple test strips positioned within the housing without moving the housing from the skin of the user. These multiple tests may be executed by one or more microprocessors such as automatically by one or more microprocessors so that one or more tests may be executed according to a programmed timing sequence without user initiation or intervention. Embodiments include advancing a skin piercer within a housing that is fixed on a skin surface of a user from a first site aligned with a first test strip to a second site to align the advanced skin piercer with a second test strip. Advantageously, such advancement may be executed automatically so that the user need not be involved.

A housing of a device as described above may be positioned in a fixed position on a skin surface of a user directly or by way of a secondary unit such as a mounting unit that is configured to receive a device. Positioning may include removing a protective liner from a surface of a device or from a mounting unit.

In certain embodiments, a meter to which the one or more test strips are connected or are connectable may be programmed to monitor for when a signal from an indicator electrode (if present) is received, thus indicating if and when sample has contacted an indicator of a test strip. Such as signal may indicate that a sufficient amount of sample has entered a sample chamber of a test strip to ensure adequate filling of a test strip, e.g., in embodiments in which an indicator electrode is downstream (closest to the meter end of a test strip than the sample filling end of the test strip) of the working electrode and the measurement zone. The signal may be an on/off signal, or may be a change (either an increase or decrease) in an existing signal.

Embodiments of the subject processes include determining the concentration of an analyte in any volume of sample, and include determining analyte concentration in a small volume of sample, e.g., a sample having a volume no more than about 1 μL, for example no more than about 0.5 μL, for example no more than about 0.25 μL, for example no more than about 0.1 μL. In some embodiments, processes include determining the concentration of an analyte a volume of sample as low as about 0.05 μL, or as low as about 0.03 μL, or less.

Analyte determination may be accomplished using any suitable technique. For example, the analyte assay may be accomplished using coulometry, amperometry and/or potentiometry or reflectometry. In certain embodiments, the measurement technique includes impedance measurement. Certain embodiments may include using photometric techniques.

A test strip may be operated with or without applying a potential to the electrodes of the strip. In one embodiment, in which the test strip is an electrochemical sensor, the electrochemical reaction may occur spontaneously and a potential need not be applied between the working electrode and counter electrode of the sensor. In another embodiment, a potential may be applied between the working electrode and counter electrode of the sensor. The potential may be constant or not, and the magnitude of the potential is dependent at least in part on the redox mediator. As above, details regarding potential as related to the sensing chemistry and the electrodes are discussed, for example, in U.S. Pat. No. 6,338,790 and elsewhere.

In certain embodiments, information related to an analyte reading (processed or not) may be forwarded (such as by communication) to a remote location if desired, and received there for further use (such as further processing). By “remote location” is meant a location other than the location at which the sample evaluation device is present and sample evaluation occurs. For example, a remote location could be another location (e.g., office, lab, etc.) in the same city, another location in a different city, another location in a different state, another location in a different country, etc. As such, when one item is indicated as being “remote” from another, what is meant is that the two items are at least in different buildings, and may be about 1 foot apart or more, e.g., about 5-10 feet apart or more, one mile, ten miles, or at least one hundred miles apart. “Communicating” information means transmitting the data representing that information as electrical signals over a suitable communication channel (for example, a private or public network). “Forwarding” an item refers to any means of getting that item from one location to the next, whether by physically transporting that item or otherwise (where that is possible) and includes, at least in the case of data, physically transporting a medium carrying the data or communicating the data. The data may be transmitted to the remote location for further evaluation and/or use. Any convenient telecommunications means may be employed for transmitting the data, e.g., facsimile, modem, Internet, etc. In some embodiments, information relating to one or more analyte tests may be communicated to a drug delivery device. Any suitable communication may be employed and includes wireless (e.g., Bluetooth, RF, IR, etc.,) or wired communication.

Finally, kits for use in practicing the subject disclosure are also provided. The subject kits may include at least one analyte testing device as described herein. Kits may include components such as a plurality of test strips and/or skin piercing members, where certain embodiments include replacement components such as replacement test strips and/or replacement skin piercing members that are used to replace used test strips and/or skin piercing members that may be removed from the device and discarded.

The kits may further include one or more additional components necessary for carrying out an analyte determination assay, such as control reagents, and the like. As such, the kits may include one or more containers such as vials or bottles, with each container containing a separate component for the assay.

In one aspect, an analyte testing device includes a housing defining an interior space comprising an axis, the interior space including: a disk configured to be held in place in a fixed position on the body of user, the disk stationary with respect to the axis and comprising: at least one test strip having a sample chamber to receive biological sample of a user; and at least one opening in the disk, the at least one opening in communication with a sample chamber of a test strip; a skin piercing member holder rotatable about the axis and comprising at least one skin piercing member alignable with an opening of the disk to pierce through the opening of the disk to the skin of a user; and a firing mechanism to advance the skin piercing member through the opening to pierce the skin of the user.

In one embodiment, the axis is a substantially central axis.

In a further embodiment, the axis is a hub.

In another embodiment, the disk is stationarilly attached to the hub and the skin piercing member holder is rotatably attached to the hub.

A further embodiment includes an automated system to test for analyte automatically according to a schedule.

In another embodiment, the automated system comprises a timing mechanism and a microprocessor, wherein the microprocessor is programmed or is programmable to control the timing and firing mechanisms to execute the automatic analyte testing according to the schedule.

In yet another embodiment, the microprocessor is programmed or is programmable to determine analyte concentration from the biological sample received by a sample chamber of the device.

The microprocessor is programmed or is programmable to align a skin piercing member with an opening of the disk in certain embodiments.

Another embodiment includes a plurality of test strips and a plurality of skin piercing members that are radially positioned about the hub.

In yet another embodiment, the hub is fixed in place.

In another embodiment, the rotatable skin piercing member holder is rotatable about the fixed hub.

In other embodiments, the microprocessor comprising programming or is programmable to rotate the skin piercing member holder in discrete steps about the hub.

In yet another embodiment, the rotatable skin piercing member holder is rotated one step for each analyte test, and each step corresponds to the distance between adjacent disk openings.

Other embodiments include a wound spring drive to drive coupled to the skin piercing member holder to drive the rotation of the rotatable skin piercing member holder.

In one embodiment, the wound spring drive energizes the skin piercing members to pierce the skin of the user.

In another embodiment, the wound spring drive provides power to the microprocessor.

In certain embodiments, the device is about 5 inches or less in diameter and has a circumference of about 10 inches or less.

In other embodiments, the device is about 2 inches or less in diameter and has a circumference of about 6.25 inches or less.

In certain embodiments, the device is able to contain about 100 test strips and about 100 skin piercing members at the same time.

In another aspect, an analyte testing device coupled to at least one microprocessor includes a housing defining an interior space comprising a hub, the interior space including: a disk comprising two or more analyte test strips, each having a sample chamber to receive biological sample of a user, and the disk having at least the same number of openings in the disk as the number of test strips, so that each test strip is in communication with a sample chamber of a test strip; a skin piercing member holder coupled to and moveable about the hub and comprising at least one skin piercing member, wherein the holder is advanceable by the one or more microprocessors to adjacent test strips so that a skin piercing member is alignable with an opening of the disk to pierce through the opening of the disk to the skin of a user; and a sterilization system to sterilize at least one skin piercing member while it is contained within the interior space.

In one embodiment, the sterilization system comprises a source of electrical current and pathway to provide the current to a skin piercing member.

In one embodiment the skin piercing member comprises a tip that comprises a conducting material, at least one contact pad to provide electrical contact between the tip and the source of electrical current, and at least one lead extending from the at least one lead to the tip.

In another embodiment the sterilization system is configured to sterilize a skin piercing member after it has been used.

In certain embodiment the device is a no calibration device such that it is configured not to require a user to take any action to calibrate the device for the two or more test strips.

In another aspect, a computer-implemented process of glucose testing includes automatically actuating a skin piercing member of an on-body analyte testing device to pierce the body to obtain a sample of biological fluid according to a timing schedule; testing the sample for glucose; and repeating the automatic actuation and testing at least one more time according to the timing schedule to perform at least two glucose tests at two time periods.

In one embodiment, the repeating comprises advancing a skin piercing member from the first glucose test to at least a second test.

In one embodiment, the skin piercing member is sterilized between the first and second glucose tests.

A further embodiment includes securing a housing of a device that comprises a power supply, at least one microprocessor, at least one skin piercing member, and at least two glucose test strips, to a body part of a user so that the housing is fixed in position on the body of a user.

Another further embodiment includes removing used or un-useable test strips from the housing and replacing useable test strips within the housing.

Yet a further embodiment includes forwarding wirelessly or by a wired connection test data from the glucose tests to an insulin pump.

In another aspect of the present disclosure, a device for piercing skin includes a housing defining an interior space; an opening in the housing from the interior space to the exterior of the housing; an assembly coupled to a frame and comprising a plurality of lancets linked together as a chain of lancets, the assembly coupled to the frame so that it is moveable about the frame as a single unit to position a lancet of the chain with respect to the opening; and a moveable carriage coupled to the assembly to move a lancet that is positioned with respect to the opening along a stroke from position wherein the lancet is substantially entirely within the interior space to an extended position wherein at least a portion of the lancet extends a distance outwardly through the opening, and back to a position within the interior space.

In one embodiment, the chain of lancets comprises a roller chain.

In a further embodiment, the device comprises at least one sprocket to which the roller chain is coupled.

In yet a further embodiment, the device comprises two sprockets.

In one embodiment, the at least one sprocket is rotatably attached to the frame.

In another embodiment, the at least one sprocket rotates about its axis to move the roller chain.

In another embodiment, the movement of the roller chain is accomplished manually.

In yet another embodiment, the movement of the roller chain is accomplished at least semi-automatically.

A further embodiment includes a selector to selectively control the stroke range.

Another further embodiment includes a lancet indicator to indicate a lancet of the chain.

In one embodiment, the indicator indicates the number of unused lancets remaining in the device or the number of used lancets in the device.

In another embodiment, the indicator is automatically re-settable each time the lancet chain is replaced with a different lancet chain.

In yet another embodiment, the indicator is manually re-settable each time the lancet chain is replaced with a different lancet chain.

In one embodiment, the chain of lancets is permanently attached to the device.

In another embodiment, the chain of lancets is removable from the device.

Certain embodiments include an analyte testing monitor within the interior space.

In certain embodiments, the analyte testing monitor is a glucose testing monitor.

In on embodiment, the glucose testing monitor is a less than about 1 microliter of sample glucose testing monitor.

In another embodiment, a plurality of test strips are retained in the interior space.

In another aspect, a method of piercing skin includes moving a chain of lancets retained within an interior space of a device so that one of the lancets is positioned proximate to an opening of the device; positioning the opening of a device proximate to a skin surface; firing the device to move the positioned lancet at least partially out of the opening to pierce skin; retracting the fired lancet into the interior space; and moving the chain of lancets to position another lancet of the chain is positioned proximate to the opening.

In one embodiment, the moving is accomplished automatically.

In another embodiment, the moving is accomplished manually.

A further embodiment includes contacting an analyte test strip to biological fluid expressed at the pierced site.

In one embodiment, the contacting comprises moving a test strip from the interior space of the housing to the biological fluid exterior of the housing.

In certain embodiments, the test strip is a less than about 1 microliter test strip.

It is evident from the above results and discussion that the above-described disclosure provides devices and processes for analyte testing, e.g., glucose testing. The above-described disclosure provides a number of advantages—some of which are described above and which include, but are not limited to, ease of use, multiple testing, automation of some or all testing tasks, and the like. As such, the subject disclosure represents a significant contribution to the art.

All publications and patents cited in this specification are herein incorporated by reference as if each individual publication or patent were specifically and individually indicated to be incorporated by reference. The citation of any publication is for its disclosure prior to the filing date and should not be construed as an admission that the present disclosure is not entitled to antedate such publication by virtue of prior disclosure.

While the present disclosure has been described with reference to the specific embodiments thereof, it should be understood by those skilled in the art that various changes may be made and equivalents may be substituted without departing from the true spirit and scope of the disclosure. In addition, many modifications may be made to adapt a particular situation, material, composition of matter, process, process step or steps, to the objective, spirit and scope of the present disclosure. All such modifications are intended to be within the scope of the claims appended hereto.

What is claimed is:

1. An analyte testing device comprising:
a housing defining an interior space comprising an axis, the interior space including:
a disk configured to be held in place in a fixed position on the body of user, the disk stationary with respect to the axis and comprising:
at least one test strip having a sample chamber to receive biological sample of a user; and
at least one opening in the disk, the at least one opening in communication with a sample chamber of a test strip;
a skin piercing member holder rotatable about the axis and comprising at least one skin piercing member alignable with an opening of the disk to pierce through the opening of the disk to the skin of a user; and
a firing mechanism to advance the skin piercing member through the opening to pierce the skin of the user.

2. The analyte testing device of claim 1, wherein the axis is a substantially central axis.

3. The analyte testing device of claim 1, wherein the axis is a hub.

4. The analyte test device of claim 2, wherein the disk is stationarilly attached to the hub and the skin piercing member holder is rotatably attached to the hub.

5. The analyte test device of claim 4, further comprising an automated system to test for analyte automatically according to a schedule.

6. The analyte test device of claim 5, wherein the automated system comprises a timing mechanism and a microprocessor, wherein the microprocessor is programmed or is programmable to control the timing and firing mechanisms to execute the automatic analyte testing according to the schedule.

7. The analyte test device of claim 6, wherein the microprocessor is programmed or is programmable to determine analyte concentration from the biological sample received by a sample chamber of the device.

8. The analyte test device of claim 4, wherein the microprocessor is programmed or is programmable to align a skin piercing member with an opening of the disk.

9. The analyte test device of claim 8, wherein device comprises a plurality of test strips and a plurality of skin piercing members that are radially positioned about the hub.

10. The analyte test device of claim 9, wherein hub is fixed in place.

11. The analyte test device of claim 10, wherein the rotatable skin piercing member holder is rotatable about the fixed hub.

12. The analyte test device of claim 11, wherein the microprocessor comprising programming or is programmable to rotate the skin piercing member holder in discrete steps about the hub.

13. The analyte test device of claim 12, wherein the rotatable skin piercing member holder is rotated one step for each analyte test, and each step corresponds to the distance between adjacent disk openings.

14. The analyte test device of claim 13, wherein the device comprises a wound spring drive to drive coupled to the skin piercing member holder to drive the rotation of the rotatable skin piercing member holder.

15. The analyte test device of claim 14, wherein the wound spring drive energizes the skin piercing members to pierce the skin of the user.

16. The analyte test device of claim 15, wherein the wound spring drive provides power to the microprocessor.

17. The analyte test device of claim 4, wherein the device is about 5 inches or less in diameter and has a circumference of about 10 inches or less.

18. The analyte test device of claim 17, wherein the device is about 2 inches or less in diameter and has a circumference of about 6.25 inches or less.

19. The analyte test device of claim 18, wherein the device is able to contain about 100 test strips and about 100 skin piercing members at the same time.

20. An analyte testing device coupled to at least one microprocessor, the analyte test device comprising:
a housing defining an interior space comprising a hub, the interior space including:
a disk comprising two or more analyte test strips, each having a sample chamber to receive biological sample of a user, and the disk having at least the same number of openings in the disk as the number of test strips, so that each test strip is in communication with a sample chamber of a test strip;
a skin piercing member holder coupled to and moveable about the hub and comprising at least one skin piercing member, wherein the holder is advanceable by the one or more microprocessors to adjacent test strips so that a skin piercing member is alignable with an opening of the disk to pierce through the opening of the disk to the skin of a user; and
a sterilization system to sterilize at least one skin piercing member while it is contained within the interior space.

21. The analyte test device of claim 20, wherein the sterilization system comprises a source of electrical current and pathway to provide the current to a skin piercing member.

22. The analyte test device of claim 21, wherein the skin piercing member comprises a tip that comprises a conducting material, at least one contact pad to provide electrical contact between the tip and the source of electrical current, and at least one lead extending from the at least one lead to the tip.

23. The analyte test device of claim 22, wherein the sterilization system is configured to sterilize a skin piercing member after it has been used.

24. The analyte test device of claim 22, wherein the device is a no calibration device such that it is configured not to require a user to take any action to calibrate the device for the two or more test strips.

25. A computer-implemented process of glucose testing, the process comprising:
automatically actuating a skin piercing member of an on-body analyte testing device to pierce the body to obtain a sample of biological fluid according to a timing schedule;
testing the sample for glucose; and
repeating the automatic actuation and testing at least one more time according to the timing schedule to perform at least two glucose tests at two time periods.

26. The computer-implemented process of glucose testing of claim 25, wherein the repeating comprises advancing a skin piercing member from the first glucose test to at least a second test.

27. The computer-implemented process of glucose testing of claim 26, wherein the skin piercing member is sterilized between the first and second glucose tests.

28. The computer-implemented process of glucose testing of claim 27, further comprising securing a housing of a device that comprises a power supply, at least one microprocessor, at least one skin piercing member, and at least two glucose test strips, to a body part of a user so that the housing is fixed in position on the body of a user.

29. The computer-implemented process of glucose testing of claim 25, further comprising removing used or un-useable test strips from the housing and replacing useable test strips within the housing.

30. The computer-implemented process of glucose testing of claim 25, further comprising forwarding wirelessly or by a wired connection test data from the glucose tests to an insulin pump.

31. A device for piercing skin, the device comprising:
a housing defining an interior space;
an opening in the housing from the interior space to the exterior of the housing;
an assembly coupled to a frame and comprising a plurality of lancets linked together as a chain of lancets, the assembly coupled to the frame so that it is moveable about the frame as a single unit to position a lancet of the chain with respect to the opening; and
a moveable carriage coupled to the assembly to move a lancet that is positioned with respect to the opening along a stroke from position wherein the lancet is substantially entirely within the interior space to an extended position wherein at least a portion of the lancet extends a distance outwardly through the opening, and back to a position within the interior space.

32. The device of claim 31, wherein the chain of lancets comprises a roller chain.

33. The device of claim 32, wherein the device comprises at least one sprocket to which the roller chain is coupled.

34. The device of claim 33, wherein the device comprises two sprockets.

35. The device of claim 33, wherein the at least one sprocket is rotatably attached to the frame.

36. The device of claim 35, wherein the at least one sprocket rotates about its axis to move the roller chain.

37. The device of claim 36, wherein the movement of the roller chain is accomplished manually.

38. The device of claim 36, wherein the movement of the roller chain is accomplished at least semi-automatically.

39. The device of claim 31, wherein the device comprises a selector to selectively control the stroke range.

40. The device of claim 31, wherein the device comprises a lancet indicator to indicate a lancet of the chain.

41. The device of claim 40, wherein the indicator indicates the number of unused lancets remaining in the device or the number of used lancets in the device.

42. The device of claim 41, wherein the indicator is automatically re-settable each time the lancet chain is replaced with a different lancet chain.

43. The device of claim 41, wherein the indicator is manually re-settable each time the lancet chain is replaced with a different lancet chain.

44. The device of claim 31, wherein the chain of lancets is permanently attached to the device.

45. The device of claim 31, wherein the chain of lancets is removable from the device.

46. The device of claim 31, wherein the device comprises an analyte testing monitor within the interior space.

47. The device of claim 46, wherein the analyte testing monitor is a glucose testing monitor.

48. The device of claim 47, wherein the glucose testing monitor is a less than about 1 microliter of sample glucose testing monitor.

49. The device of claim 47, wherein a plurality of test strips are retained in the interior space.

50. A method of piercing skin, the method comprising:
moving a chain of lancets retained within an interior space of a device so that one of the lancets is positioned proximate to an opening of the device;
positioning the opening of a device proximate to a skin surface;
firing the device to move the positioned lancet at least partially out of the opening to pierce skin;
retracting the fired lancet into the interior space; and
moving the chain of lancets to position another lancet of the chain is positioned proximate to the opening.

51. The method of claim 50, wherein the moving is accomplished automatically.

52. The method of claim 50, wherein the moving is accomplished manually.

53. The method of claim 50, further comprising contacting an analyte test strip to biological fluid expressed at the pierced site.

54. The method of claim 53, wherein the contacting comprises moving a test strip from the interior space of the housing to the biological fluid exterior of the housing.

55. The method of claim 54, wherein the test strip is a less than about 1 microliter test strip.
